(19) United States
(12) Patent Application Publication
(10) Pub. No.: US 2021/0130876 A1
(43) Pub. Date: May 6, 2021

(54) METHOD OF IMPROVING ELECTROCHEMILUMINESCENCE SIGNAL IN BIOANALYTICAL ASSAYS

(71) Applicant: Rubipy Scientific Inc., Greely (CA)

(72) Inventor: Ming Zhou, Ontario (CA)

(21) Appl. No.: 16/670,226

(22) Filed: Oct. 31, 2019

(51) Int. Cl. C12Q 1/6825; C12Q 1/00; C12Q 1/6869

(52) U.S. Cl. CPC C12Q 1/6825; C12Q 1/6869; C12Q 1/005

(57) ABSTRACT

The present invention relates to a method for bioanalytical assays, more specifically, immunoassays and nucleic acid assays using electrogenerated chemiluminescence or electrochemiluminescence (ECL) of delocalized luminophores.

### BACKGROUND OF THE INVENTION

### 1. Field of Invention

The present invention relates to improvement in bioanalytical assays, more specifically, immunoassays and nucleic acid assays using electrogenerated chemiluminescence or electrochemiluminescence (ECL) of delocalized luminophores.

### 2. Description of Related Art

Affinity-based bioanalytical assays, such as immunoassay and DNA probing, rely largely on labeling technique, by which signal-generating units are covalently bound to certain functional moieties of biomolecules that specifically bind to the analytes in assay protocols. In some competitive assay formats, a signal-generating unit is covalently linked to an analyte molecule, which competes with the analyte in sample for the analyte's binding partner. In clinical electrochemiluminescence (ECL) immunoassay, an active ester of ruthenium(II) trisbipyridine complex, such as [4-(N-succimidyl-oxycarbonylpropyl)-4′-methyl-2,2′-bipyridine]bis-(2,2′-bipyridine) ruthenium (II) dihexafluorophosphate (FIG. 1A), serves as a labeling molecule (U.S. Pat. No. 5,744,367 and G. F. Blackburn et al., Clin. Chem. 1991, 37/9, 1534-1539). Upon the electrochemical oxidation of the luminophore [Ru(bpy)₃]²⁺ and a cascade of downstream chemical reactions of the co-reactant tri-n-propylamine (TPA), a luminescent excited state Ru(bpy)₃²⁺* is formed and emits detectable luminescence. A simplified reaction sequence is demonstrated in the following Scheme 1 (see J. K. Leland and M. J. Powell, J. Electrochem. Soc. 1990, 137, 3127-3131).

### Scheme 1

Ru(bpy)₃²⁺−e⁻→Ru(bpy)₃³⁺ (1)

N(C₃H₇)₃−e⁻→N(C₃H₇)₃^{*+} (2)

N(C₃H₇)₃^{*+}−H⁺→H₆C₃*N(C₃H₇)₂ (3)

Ru(bpy)₃³⁺+H₆C₃*N(C₃H₇)₂→Ru(bpy)₃^{2+*}+P (4)

Ru(bpy)₃²⁺*→Ru(bpy)₃²⁺+hv (5)

There are many technical details in an ECL immunoassay, which involves how an antibody (in a sandwich assay) or an analyte (in a competitive assay) is labeled, how the anaylate is captured, how the ECL reactions are triggered and how the working electrode is regenerated etc. Taking an ECL sandwich immunoassay as an example, in a typical commercial ECL assay, an antibody (signaling antibody) is labeled with a number of Ru(bpy)₃²⁺ luminophores using one of the label molecules shown in FIG. 1 at the ε-amino sites of lysine residues of the antibody and another antibody (capturing antibody) is biotinylated. When a clinical sample is mixed with the two types of antibodies and the streptavidin-coated magnetic microbeads for a pre-determined period of time, a sandwich immunocomplex is formed on the surface of the microbeads. The microbeads are then brought into an ECL measuring cell (a flow cell) and are captured on the surface of the working electrode by a movable magnet underneath. A TPA containing buffer washes out the undesired substances and provides a chemical environment for Ru(bpy)₃²⁺ luminophore to undergo the ECL reactions described in Scheme 1.

At a specific potential (e.g., 1.4 V vs. Ag/AgCl), Ru(bpy)₃²⁺ and TPA are concomitantly oxidized to become Ru(bpy)₃³⁺ and a cation radical N(C₃H₇)₃⁻⁺ (Reactions 1 and 2). The latter is unstable and quickly loses a proton to become a neutral radical H₆C₃⁺N(C₃H₇)₂ (Reaction 3). The neutral radical has a strong reducing ability and reduces Ru(bpy)₃³⁺ to its luminescent state Ru(bpy)₃^{2+*} (Reaction 4). It is Ru(bpy)₃^{2+*} that emits light at 620 nm wavelength and, upon emission, decays to the original ground state Ru(bpy)₃²⁺ (Reaction 5). Therefore, Ru(bpy)₃²⁺ is not consumed in the ECL process but, as disclosed in U.S. Pat. No. 6,165,708, undergoes a cycle of the oxidation state change, i.e., Ru(bpy)₃²⁺ [symbol] Ru(bpy)₃³⁺ [symbol] Ru(bpy)₃^{2+*} [symbol] Ru(bpy)₃²⁺. The cycle keeps repeating itself during the period of measurement and a long-lasting ECL signal can be generated and measured. The integration of the total ECL emission over a certain period of time (e.g., from 0.5 to 5 seconds) can be a measure of the ECL intensity and can be correlated to the quantity of the analyte. After the measurement, the microbeads and the attached immunocomplex are washed away by aqueous flow and the measuring cell is cleaned and the electrode surface is electrochemically regenerated for the next sample. The sequential experimental details were disclosed in the U.S. Pat. Nos. 5,538,687 and 6,599,473B1.

As a matter of fact, Scheme 1 is just one reaction scheme that leads to ECL. Other possible pathways (Schemes 2-4 below) have been proposed (see J. K. Leland and M. J. Powell, J. Electrochem. Soc. 1990, 137, 3127-3131, and W. Miao, J.-P. Choi, A. J. Bard, J. Am. Chem. Soc. 2002, 124, 14478-14485) to elucidate the formation of excited state Ru(bpy)₃^{2+*} or the generation of ECL under different conditions.

### Scheme 2

Ru(bpy)₃²⁺−e⁻→Ru(bpy)₃³⁺ (1)

Ru(bpy)₃³⁺+N(C₃H₇)₃→Ru(bpy)₃²⁺+N(C₃H₇)₃^{*+} (6)

N(C₃H₇)₃^{*+}−H⁺→H₆C₃^{*}N(C₃H₇)₂ (3)

Ru(bpy)₃³⁺+H₆C₃^{*}N(C₃H₇)₂→Ru(bpy)₃²⁺*+P (4)

Ru(bpy)₃²⁺*→Ru(bpy)₃²⁺+hv (5)

### Scheme 3

Ru(bpy)₃²⁺−e⁻→Ru(bpy)₃³⁺ (1)

N(C₃H₇)₃−e⁻→N(C₃H₇)₃^{*+} (2)

N(C₃H₇)₃^{*+}−H⁺→H₆C₃^{*}N(C₃H₇)₂ (3)

Ru(bpy)₃²⁺+H₆C₃^{*}N(C₃H₇)₂→Ru(bpy)₃⁺P (7)

Ru(bpy)₃⁺+Ru(bpy)₃³⁺→Ru(bpy)₃²⁺+Ru(bpy)₃²⁺* (8)

Ru(bpy)₃²⁺*→Ru(bpy)₃²⁺+hv (5)

### Scheme 4

N(C₃H₇)₃−e⁻→N(C₃H₇)₃^{*+} (2)

N(C₃H₇)₃^{*+}−H⁺→H₆C₃^{*}N(C₃H₇)₂ (3)

Ru(bpy)₃²⁺+H₆C₃^{*}N(C₃H₇)₂→Ru(bpy)₃⁺P (7)

Ru(bpy)₃⁺+N(C₃H₇)₃^{*+}→Ru(bpy)₃^{2+*}+N(C₃H₇)₃ (9)

Ru(bpy)₃²⁺*→Ru(bpy)₃²⁺+hv (5)

It must be pointed out that in a sandwich immunoassay, the Ru(bpy)₃²⁺ luminophores are attached to an antibody (so-called signaling antibody) and through the antibody/antigen/antibody immunocomplex, are immobilized on the solid phase surface of the microbeads or microwell plate. Therefore, all reactions involving a ruthenium complex, i.e., Ru(bpy)₃²⁺, Ru(bpy)₃²⁺ or Ru(bpy)₃³⁺ are heterogeneous reactions in immunoassay. No matter how the excited state is formed, the light is emitted from the solid phase surface (U.S. Pat. No. 6,881,589B1) rather than from the solution phase. Immobilization or localization limits the accessibility of ruthenium complexes to other reactive species, i.e., the TPA-derived intermediates. Despite the proposals for improving ECL signals based on many other methods or techniques, such as novel ECL luminophores (U.S. Pat. No. 6,808,939B2, WO2014203067A1, WO2014019711A1), co-reactants (X. Liu et al, Angew. Chem. Int. Ed. 2007, 46, 421-424, WO2017153574A1), multilabeling at a single-site (US 2005/0059834 A1 and CA 2481982 A1; M. Zhou et al., Anal. Chem. 2003, 75, 6708-6717) etc., the obviously unfavorable outcome as a result of immobilization or localization of Ru(bpy)₃²⁺ luminophore remains unaddressed since the ECL was employed as a mainstream immunoassay platform technology. An optimal or ideal environment for the ECL reactions to occur would be the one in which the ruthenium complex luminophores can freely access the TPA radical cation N(C₃H₇)₃^{*+} and/or the TPA neutral radical H₆C₃⁺N(C₃H₇)₂ in the solution phase. That is to say, in order for them to reach the highest reaction efficiencies, ruthenium complex units Ru(bpy)₃⁺, Ru(bpy)₃²⁺ or Ru(bpy)₃³⁺ must be detached from the labeled species (e.g., the signaling antibody in sandwich immunoassay) and thus be released from their immobilized state on the solid surface to become free in solution phase.

This present invention provides a method of releasing (detaching, delocalizing or liberating) the bound ECL luminophores, so that the excited state, such as Ru(bpy)₃^{2+*}, and thus the ECL could be generated in solution (homogeneous) phase to enhance signal intensity.

### BRIEF SUMMARY OF THE INVENTION

The present invention provides a means to release the ECL luminophores from the localized or immobilized state in bioanalytical assays. With the delocalized luminophores in homogeneous solution phase, rather than being immobilized on the solid phase surface, the ECL signal can be enhanced. The ECL luminophores described in the present invention were exemplified with ruthenium(II) complexes. However, other ECL luminophores, such as organic compounds and metal complexes containing osmium, platinum, rhenium, iridium etc., are also contemplated.

### BRIEF DESCRIPTION OF SEVERAL VIEWS OF THE DRAWINGS

The invention will be described in conjunction with the following drawings in which like reference numerals designate like elements and wherein:

FIGS. 1A, 1B, 1C, and 1D show ECL label molecules disclosed in U.S. Pat. No. 5,744,367 (A), U.S. Pat. No. 6,808,939 (B), WO 2014203067A1 (C, used as a reference label in this invention and denoted as Ref)), and U.S. patent application US 2016/0145281A1 (D);

FIG. 2 illustrates electrochemical dealkylation of tri-n-propylamine producing secondary amine (CH₃CH₂CH₂)₂NH;

FIGS. 3A, 3B, 3C, and 3D illustrate four examples of ECL labels of this invention—(A) Cationic label with two ruthenium(II) complex luminophores linked to an amine N-center; (B) Anionic label with two ruthenium(II) complex luminophores linked to an amine N-center; (C) Electronically neutral label with two ruthenium(II) complex luminophores linked to an amine N-center; (D) Electronically neutral label with two iridium(III) complex luminophores linked to an amine N-center. The circled moieties are ECL luminophores as defined within the scope of this invention.

FIG. 4 illustrates the present invention used in a sandwich immunoassay;

FIG. 5 illustrates using electrochemical dealkylation to release fragments containing ECL luminophore(s) from the labeled antibodies that is immobilized on the microbeads during the course of ECL generation in immunoassay;

FIGS. 6A and 6B illustrate the present invention used in competitive immunoassays—(A) the analyte in a sample competing with the labeled analyte; (B) the analyte in a sample competing with the biotinylated analyte.

FIG. 7 are examples of ECL labels with the hierarchically different N-centers bearing multiple ruthenium(II) complex luminophores;

FIG. 8 are examples of the possible fragments containing ECL luminophores that could be released from a label with hierarchically different N-centers according to the present invention;

FIG. 9 is an illustration of a preparation of a label with two ruthenium complex luminophores according to one embodiment of the current invention;

FIG. 10 is an illustration of a convergent synthetic approach to polyamine dendritic labels with multiple ruthenium(II) complex luminophores at the periphery;

FIG. 11 is a graph of the ECL signal generated by detached ruthenium complex luminophores from sandwich immunocomplex loaded magnet beads 20 (antibody labeled with 14). Indicated are antigen concentration.

FIG. 12 is a graph of the ECL signals generated by detached ruthenium complex luminophores from sandwich immunocomplex loaded magnet beads 20 (antibody labeled with 14), and by the immobilized ruthenium complex luminophores (antibody labeled with Ref).

### DETAILED DESCRIPTION OF PREFERRED EMBODIMENTS OF THE INVENTION

The practicing of the methods described herein will typically include, unless otherwise indicated, conventional techniques of molecular biology (including recombinant techniques), microbiology, cell biology, biochemistry, antibody engineering, and immunology, which are within the skill of the art. Such techniques are explained fully in the literature, such as, “Bioconjugate Techniques” (G. T. Hermanson, Elservier, Amsterdam, 2008), “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Unless defined otherwise, technical and scientific terms used herein have the same meaning as commonly understood by one of ordinary skill in the art to which the methods, reagent compositions and kits described herein belong. Wild, D et al. (eds.), The Immunoassay Handbook, 4th ed., Elservier, Amsterdam (2013), Singleton et al., Dictionary of Microbiology and Molecular Biology, 2nd ed., J. Wiley & Sons, New York (1994); March, Advanced Organic Chemistry Reactions, Mechanisms and Structure, 4th ed., John Wiley & Sons, New York (1992); Lewin, B., Genes V, published by Oxford University Press (1994), ISBN 0-19-854287 9); Kendrew, J. et al. (eds.), The Encyclopedia of Molecular Biology, published by Blackwell Science Ltd. (1994), ISBN 0-632-02182-9); and Meyers, R. A. (ed.), Molecular Biology and Biotechnology: a Comprehensive Desk Reference, published by VCH Publishers, Inc. (1995), ISBN 1-56081-5698) provide one skilled in the art with a general guidance to many of the terms used in the present application. All references cited herein, including patent applications and publications, are incorporated by reference in their entirety.

Herein, the term “detach”, “delocalize”, “release”, “liberate” and the gerund form (“-ing” form) or noun form thereof are sometime used interchangeably. Within the scope of the invention, detaching (delocalizing, releasing or liberating) a “luminophore” or “ECL luminophore” or “ruthenium complex luminophore” means transforming a light-generating unit from its immobilized state on solid surface to a mobile or a free state in homogeneous solution.

It is within the scope of the invention for the species termed “label”, “label molecule”, “ruthenium(II) label” and “ECL label” to be covalently bonded to other substances such as a biologically active analyte or an analog thereof, an affinity-based recognition partner of the analyte or an analog thereof (such as an analyte specific reagent), and further binding partners of such aforementioned recognition partner, or a reactive chemical capable of forming covalent bond with the analyte, an analog thereof or a binding partner as mentioned above. The above-mentioned species can also be linked to a combination of one or more binding partners and/or one or more reactive components. Additionally, the aforementioned species can also be linked to an analyte or its analog bound to a binding partner, a reactive component, or a combination of one or more binding partners and/or one or more reactive components. It is also within the scope of the invention for a plurality of the aforementioned species to be bound directly, or through other molecules as discussed above, to an analyte or its analog.

The term “label” as used herein refers to any chemical or biochemical substance that yields, by itself or through physical/chemical interaction with other reagents, detectable signals (whether visibly or by using suitable instrumentation) that could be correlated to the quantity of the analytes of interest. Labels include, but are not limited to, molecules containing radioactive atom(s) (radioactivity), luminescent compounds (emitting light under photoexcitation or by chemical reactions), electroactive compounds (generating electronic signal through redox reactions), magnetic particles (magnetic signal), enzymes (generating detectable species or optical signal via the reaction with substrates), enzymes or enzymatic substrates (catalyzing chemical/biochemical reactions). A label may be composed of one or more signal generating unit(s) and one or more reactive group(s). The latter readily form covalent bond(s) with chemical or biochemical molecules to be labeled.

Analytes that may be measured include, but are not limited to, whole cells, cell surface antigens, protein complexes, cell signaling factors and/or components, second messengers, second messenger signaling factors and/or components, subcellular particles (e.g., organelles or membrane fragments), viruses, prions, dust mites or fragments thereof, viroids, immunological factors, antibodies, antibody fragments, antigens, haptens, fatty acids, nucleic acids (and synthetic analogs), ribosomes, proteins (and synthetic analogs), lipoproteins, polysaccharides, inhibitors, cofactors, haptens, cell receptors, receptor ligands, lipopolysaccharides, glycoproteins, peptides, polypeptides, enzymes, enzyme substrates, enzyme products, nucleic acid processing enzymes (e.g., polymerases, nucleases, integrases, ligases, helicases, telomerases, etc.), protein processing enzymes (e.g., proteases, kinases, protein phophatases, ubiquitin-protein ligases, etc.), cellular metabolites, endocrine factors, paracrine factors, autocrine factors, cytokines, hormones, pharmacological agents, drugs, therapeutic drugs, synthetic organic molecules, organometallic molecules, tranquilizers, barbiturates, alkaloids, steroids, vitamins, amino acids, sugars, lectins, recombinant or derived proteins, biotin, avidin, streptavidin, or inorganic molecules present in the sample.

An “analyte specific reagent” (ASR) according to the present methods and reagents has to be understood as a molecule or biomolecule (e.g., a protein or antibody) with the capability to specifically bind the analyte. “Analyte specific reagents” (ASRs) are a class of biological molecules which can be used to identify and measure the amount of an individual chemical or biochemical substance in biological specimens. ASRs are, for example, antibodies, both polyclonal and monoclonal, specific receptor proteins, ligands, nucleic acid sequences, and similar reagents which, through specific binding or chemical reaction with substances in a specimen, are intended for use in a diagnostic application for identification and quantification of an individual chemical or biochemical substance or ligand in biological specimens. In simple terms an ASR is the active ingredient of an assay. An ASR will fulfill both the criteria for affinity as well as for specificity of binding the analyte.

A “detection reagent” according to the present application comprises an analyte specific reagent (ASR) labeled with at least two ECL luminophores, or an analyte analog/homolog labeled with at least two ECL luminophores. According to the test format, it is known to the skilled artisan, which detection reagent has to be selected for the various assay formats (e.g., sandwich assay, double antigen bridging assay (DAGS), competitive assay, homogeneous assay, heterogeneous assay). A detection reagent in a heterogeneous immunoassay might be for example an antibody labeled with at least two ECL luminophores. It is known to a person skilled in the art that the detection reagent is eventually immobilized on a solid phase in the assay. In an embodiment the method for measuring an analyte in a sample via ECL detection can be performed as a homogeneous assay. In an embodiment the method can be performed as a heterogeneous assay. In an embodiment the method can be performed in a sandwich assay format. In an embodiment the method can be performed in a competitive assay format. Also in an embodiment the method can be performed in a double antigen bridging assay format (DAGS). Known immunoassay formats are described in detail in the books of C. P. Price and D. J. Newman, Principles and Practice of Immunoassay, 2nd ed., Stockton Press, New York (1997), D. Wild et al. (eds.), The Immunoassay Handbook, 4th ed., Elservier, Amsterdam (2013), and E. P. Diamondis and T. K. Christopoulos, Immunoassay, Academic Press, San Diego (1996).

The term “luminescence” refers to any emission of light that does not derive energy from the temperature of an energy source (for example, a source of electromagnetic radiation, a chemical reaction, mechanical energy). In general, the source causes an electron of an atom to move from a lower energy state into an “excited” higher energy state; then the electron releases that energy in the form of emitted light when it falls back to a lower energy state. Such emission of light usually occurs in the visible or near-visible range of the electromagnetic spectrum. The term “luminescence” includes, but is not limited to, such light emission phenomena as phosphorescence, fluorescence, bioluminescence, radioluminescence, electroluminescence, electrochemiluminescence and thermo-luminescence.

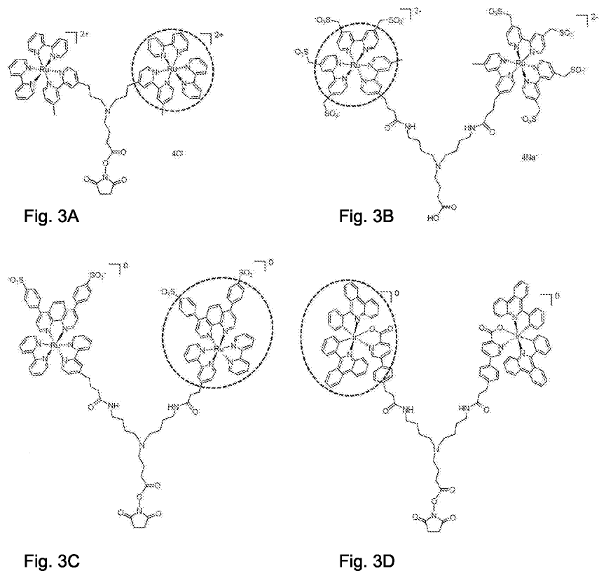

Within the scope of the invention, the term “luminophore” refers to a functional group in a chemical compound that is responsible for the generation of luminescence. In a compound with a complex structure, e.g., a structure with multiple functional groups (e.g., reactive group, hydrophilic/hydrophobic/amphiphilic group, electron withdrawing/donating group, electricity balancing group, spacing group, linking group, branching group etc.), the luminophore is the minimum structural moiety (see, for example, the circled moieties in FIG. 3) that is required for the generation of luminescence.

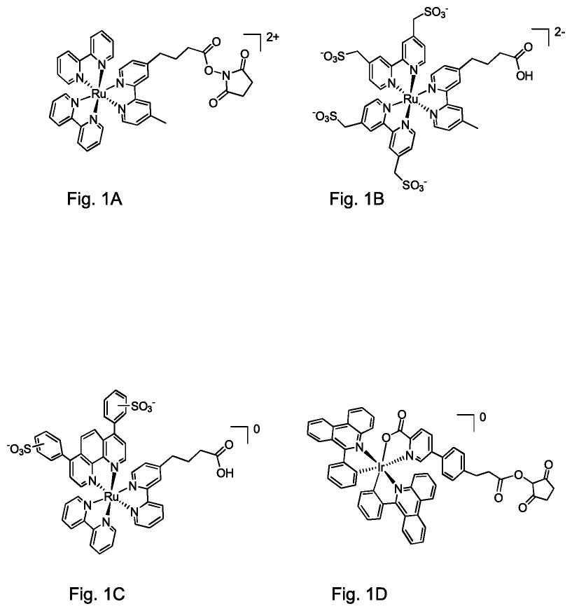

The term “luminescent label” refers to a label that is composed of one or more luminophore(s) and one or more reactive group(s), which readily form covalent bond(s) with chemical or biochemical molecules to be labeled. The luminescent label may be, for example, a fluorescent molecule, a phosphorescent molecule, a radioluminescent molecule, an electrochemiluminescent molecule (i.e., an ECL label) in the present invention, or a quantum dot with reactive groups on the dot surface. Examples of electrochemiluminescent (ECL) labels with one luminophore and one reactice group were most frequently disclosed in prior art (see, for example, the labels in FIG. 1 and other ruthenium complex labels in WO2003002974A2, WO 2014203067A1 and iridium complex labels WO2014019711A1). An example of luminescent label with three luminophores (three ruthenium complex units) and one reactive group (—COOH or NHS ester) was disclosed in US 2005/0059834 A1. An example of luminescent label with one luminophores (one ruthenium complex) and two reactive group (—COOH or NHS ester) was disclosed in U.S. Pat. No. 6,140,138.

An “electrochemiluminescence assay” or “ECL assay” is an assay in which the luminescent signal is electrochemically generated from an ECL luminophore. A voltage between a working electrode and a reference electrode electrochemically initiates luminescence from an ECL luminophorebound to an ASR or an analyte analog/homolog. Light emitted from the ECL luminophoreis measured by a photodetector and indicates the presence or quantity of an analyte of interest. ECL methods are described, for example, in U.S. Pat. Nos. 5,543,112; 5,935,779; and 6,316,607. Signal modulation can be maximized for different analyte concentrations for precise and sensitive measurements.

In an ECL assay procedure, microparticles can be suspended in the sample to efficiently bind to the analyte. For example, the particles can have a diameter of 0.05 μm to 200 μm, 0.1 μm to 100 μm, or 0.5 μm to 10 μm, and a surface component capable of binding an analyte molecule. In one frequently used ECL assay system (Cobas®, Roche Diagnostics, Germany), the microparticles have a diameter of about 3 μm. The microparticles can be formed of crosslinked starch, dextran, cellulose, protein, organic polymers, polystyrene, styrene copolymer such as styrene/butadiene copolymer, acrylonitrile/butadiene/styrene copolymer, vinylacetyl acrylate copolymer, vinyl chloride/acrylate copolymer, inert inorganic materials, chromium dioxide, oxides of iron, silica, silica mixtures, proteinaceous matter, or mixtures thereof, including but not limited to sepharose beads, latex beads, core-shell particles, and the like. The microparticles are typically monodisperse, and can be magnetic, such as paramagnetic beads. See, for example, U.S. Pat. Nos. 4,628,037; 4,965,392; 4,695,393; 4,698,302; and 4,554,088. Microparticles can be used in an amount ranging from about 1 to 10,000 μg/ml, typically 5 to 1,000 μg/ml.

A “reagent composition” or “ECL-reagent composition” according to the present application comprises reagents supporting ECL-signal generation, e.g., a coreactant, a buffering agent for pH control, a surfactant, a preservative or antibacterial agent, and optionally other components. The skilled artisan is aware of components to be present in a reagent composition which are required for ECL signal generation in electrochemiluminescent detection methods.

An “aqueous solution” as used herein is a homogeneous solution of particles, substances or liquid compounds dissolved in water, or a heterogeneous suspension with microparticles (diameter ranging from 0.05 μm to 200 μm) suspended in water solution. An aqueous solution may also comprise organic solvents. Organic solvents are known to the person skilled in the art, e.g., amines, methanol, ethanol, dimethylformamide or dimethylsulfoxide. As used herein it is also to be understood that an aqueous solution can comprise at most 50% organic solvents.

A species that participates with the ECL label in the ECL process is referred to herein as ECL “coreactant”. Commonly used coreactants for ECL include tertiary amines (e.g., tri-n-propylamine (TPA)) and its analogs/homologs (e.g., 2-(dibutylamino)ethanol etc.), oxalate, and persulfate. The skilled artisan is aware of available coreactants useful for ECL detection methods.

A “solid phase”, also known as “solid support”, is insoluble, functionalized, polymeric or non-polymeric material to which library members or reagents may be attached or covalently bound (often via a linker) to be immobilized or allowing them to be readily separated (by filtration, centrifugation, washing etc.) from excess reagents, soluble reaction by-products, or solvents. Solid phases for the methods described herein are widely described in the state of the art (see, e.g., J. E. Butler, Methods, 2000, 22, 4-23). The term “solid phase” means a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes, chips or other spectrometer sample containers. A solid phase component of an assay is distinguished from inert solid surfaces with which the assay may be in contact in that a “solid phase” contains at least one moiety on its surface, which is intended to interact with the capturing antibody or capturing molecule. A solid phase may be a stationary component, such as a tube, strip, cuvette, chip or microtiter plate, or may be non-stationary components, such as beads and microparticles. Microparticles can also be used as a solid phase for homogeneous assay formats. A variety of microparticles that allow either non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example C. R. Martin, et al., Analytical Chemistry-News & Features, 1998, 70, 322A-327A, which is incorporated herein by reference.

The “transition metal complex”, as used herein, relates to a ECL luminophore comprising a transition metal ion complexed with appropriate complexing agents. In an embodiment, the transition metal is selected from the group consisting of ruthenium (Ru), iridium (Ir), rhenium (Rh), osmium (Os), europium (Eu), terbium (Te), and dysprosium (Dy); in a further embodiment, the transition metal is ruthenium, iridium, rhenium, or osmium; in a further embodiment, the transition metal is ruthenium or iridium.

As an illustration, the ruthenium (II) or iridium(III) complexes are represented by a general formula, as commonly seen in prior art (WO 2003002974, WO 2014203067 A1, WO2014019711A1) and literature.

Mⁿ⁺[L¹ L² L³]ⁿ⁻

wherein M is ruthenium (II) or iridium(III); L¹, L², and L³, same or different, are the complexing agent, independently selected from nitrogen-containing heterocyclic bidentate ligands (2,2′-bipyridines, 1,10-phenanthrolines and their substituted analogs) and cyclometallating ligands (phenylpyridine, phenylquinoline, phenylphenanthridine, pyridine-2-carboxylic acid and analogs/derivatives thereof), and at least one of the L¹, L², and L³ is covalently linked to a functional moiety of a molecule.

Transition metal complex ECL luminophores bearing reactive, or bioconjugatable, groups are for example disclosed in WO 8706706 A1, WO 2003002974, EP720614(A1) and WO 2014203067 A1. In an embodiment, the ECL luminophore is selected from cationic ECL labels disclosed in prior art. For example, bis-(2,2′-bipyridine) [4-(N-succimidyl-oxycarbonylpropyl)-4′-methyl-2,2′-bipyridine] ruthenium (II), Ru(bpy)₂-bpyCO-OSu, which is the N-hydroxysuccinimide ester of Ru(bpy)2-bpyCOOH (CAS Reg. No. 115239-59-3), bis[(4,4′-carbomethoxy)-2,2′-bipyridine]-2-[3-(4-methyl-2,2′-bipyridine-4-yl)propyl]-1,3-dioxolane ruthenium (II); bis(2,2′-bipyridine)[4-(butan-1-al)-4′-methyl-2,2′-bipyridine] ruthenium (II); bis(2,2′-bipyridine)[4-(4′-methyl-2,2′-bipyridine-4′-yl)-butyric acid] ruthenium (II), bis(2,2′-bipyridine)[1-bromo-4-(4′-methyl-2,2′-bipyridine-4-yl)-butane] ruthenium (II), bis(2,2′-bipyridine) maleimido-hexanoic acid, 4-methyl-2,2′-bipyridine-4′-butylamide ruthenium (II).

As aforesaid, a luminophore is the minimum structural moieties that is required for the generation of luminescence. An ECL luminophore, within the scope of the invention, is the minimum structural moiety (see, for example, the circled moieties in FIG. 3) that is required for the ECL generation under electrochemical excitation. It is known to a person skilled in the art that various hydrophilic/hydrophobic/amphiphilic group, electron withdrawing/donating group, electricity balancing group, spacing group, linking group, branching group etc. can be incorporated into the complexing agents L¹, L², and L³. Therefore, in a further embodiment, the ruthenium complex ECL luminophores can be modified with one or more substituent group(s) on the complexing agents L¹ and/or L² and/or L³ (see below structures).

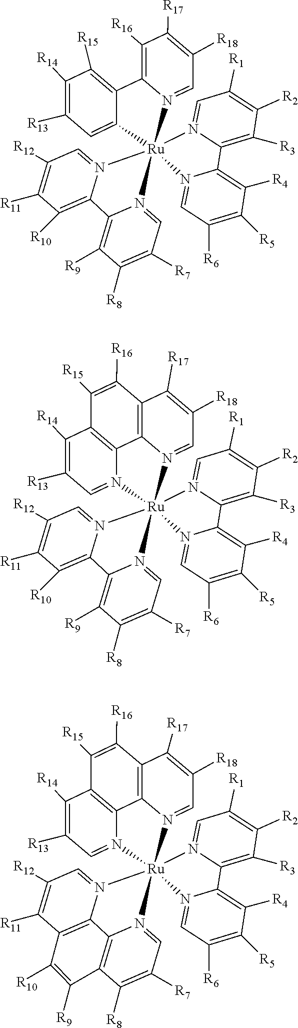

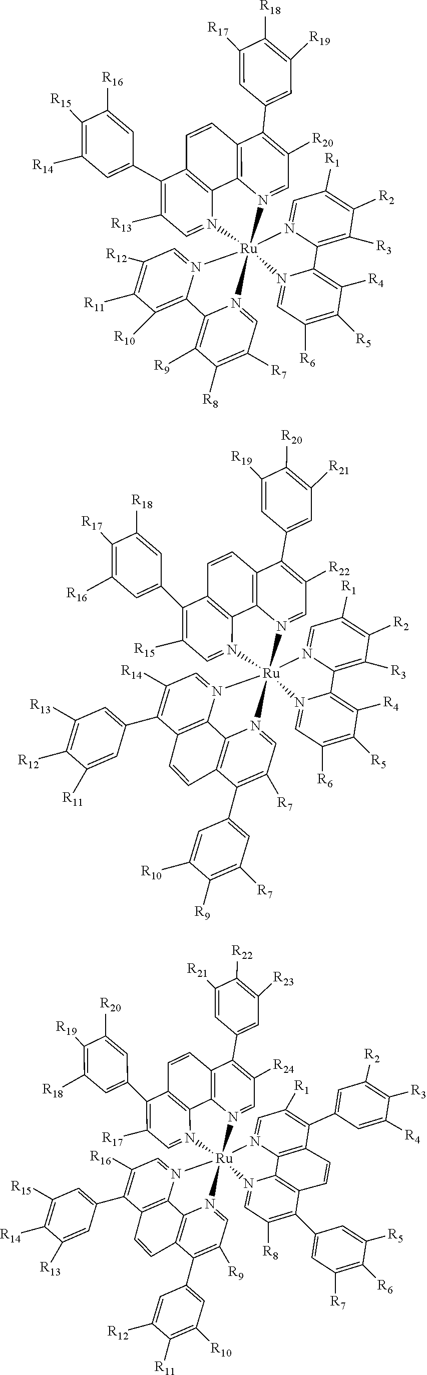

wherein R₁-R₂₄ is hydrogen, halide, cyano- or nitro-group, amino, alkylamino, substituted alkylamino, arylamino, substituted arylamino, alkylammonium, substituted alkylammonium, carboxy, carboxylic acid ester, carbamoyl, hydroxy, substituted or unsubstituted alkyloxy, substituted or unsubstituted aryloxy, sulfanyl, alkylsulfonyl, arylsulfonyl, sulfo, sulfino, sulfeno, sulfonamide, sulfoxide, sulfodioxide, phosphonate, phosphinate or R₂₅, wherein R₂₅ is aryl, substituted aryl, alkyl, substituted alkyl, branched alkyl, substituted branched alkyl, arylalkyl, substituted arylalkyl, alkylaryl, substituted alkylaryl, alkenyl, substituted alkenyl, alkynyl, substituted alkynyl, wherein the substituent is selected from hydrogen, halide, cyano- or nitro-group, a hydrophilic group, like amino, alkylamino, substituted alkylamino, alkylammonium, substituted alkylammonium, carboxy, carboxylic acid ester, carbamoyl, hydroxy, substituted or unsubstituted alkyloxy, substituted or unsubstituted aryloxy, sulfanyl, alkylsulfonyl, arylsulfonyl, sulfo, sulfino, sulfeno, sulfonamide, sulfoxide, sulfodioxide, phosphonate, phosphinate.

In a further embodiment, the ECL luminophore is an iridium complex and is selected from the below ECL labels. Ir(6-phenylphenanthridine)₂-pyridine-2-carboxylic acid or a derivative thereof, including, e.g. Ir(6-phenylphenanthridine)₂-3-Hydroxypyridine-2-carboxylic acid, Ir(6-phenylphenanthridine)₂-4-(Hydroxymethyl)pyridine-2-carboxylic acid, Ir(6-phenylphenanthridine)₂-2-(Carboxyethyl-phenyl)pyridine-2-carboxylic acid Ir(6-phenylphenanthridine)₂-5-(Methoxy)pyridine-2-carboxylic acid, or an Ir(6-phenylphenanthridine)₂-2-(Carboxyethyl-phenyl)pyridine-2-carboxylic acid ester, or derivatives of it like iridium complexes with ligands substituted with one or more sulfonic acids or iridium complexes as described in WO2012107419 (A1), WO2012107420 (A1), WO2014019707 (A2), WO2014019708 (A1), WO2014019709 (A2), WO2014019710 (A1), WO2014019711 (A1). It is well known to a person skilled in the art that iridium(III) complexes have poor solubility in aqueous solutions and hydrophilic derivatives of the aforesaid ECL compounds can be used. Therefore in a further embodiment, the aforesaid iridium(III) ECL luminophores can be modified with hydrophilic substituent group(s). In an further embodiment the ECL luminophore is an iridium complex with two phenylphenanthridine ligands having two sulfonatopropoxy substituents, two sulfo-methyl, comprising 2,9-phenanthridinedimethanesulfonic acid, 6-phenyl-, sodium salt (CAS Registry Number 1554465-50-7) or two polyethylenglycol substituents, or three of each, or combinations thereof.

These and other iridium complex ECL luminophores modified with one or more substituent group(s) are, for example, presented in the structures below (see US 2016/0145281A1).

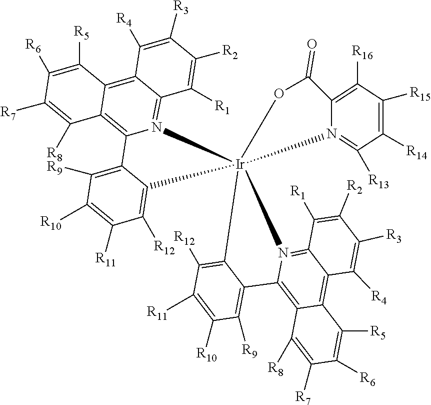

wherein R₁-R₁₆ is hydrogen, halide, cyano- or nitro-group, amino, alkylamino, substituted alkylamino, arylamino, substituted arylamino, alkylammonium, substituted alkylammonium, carboxy, carboxylic acid ester, carbamoyl, hydroxy, substituted or unsubstituted alkyloxy, substituted or unsubstituted aryloxy, sulfanyl, alkylsulfonyl, arylsulfonyl, sulfo, sulfino, sulfeno, sulfonamide, sulfoxide, sulfodioxide, phosphonate, phosphinate or R₁₇, wherein R₁₇ is aryl, substituted aryl, alkyl, substituted alkyl, branched alkyl, substituted branched alkyl, arylalkyl, substituted arylalkyl, alkylaryl, substituted alkylaryl, alkenyl, substituted alkenyl, alkynyl, substituted alkynyl, wherein the substituent is selected from hydrogen, halide, cyano- or nitro-group, a hydrophilic group, like amino, alkylamino, substituted alkylamino, alkylammonium, substituted alkylammonium, carboxy, carboxylic acid ester, carbamoyl, hydroxy, substituted or unsubstituted alkyloxy, substituted or unsubstituted aryloxy, sulfanyl, alkylsulfonyl, arylsulfonyl, sulfo, sulfino, sulfeno, sulfonamide, sulfoxide, sulfodioxide, phosphonate, phosphinate.

The present invention provides a method of electrochemically liberating the ECL luminophores or the fragments containing one or more ECL luminophore(s) from the immobilized state before or during the ECL generation process.

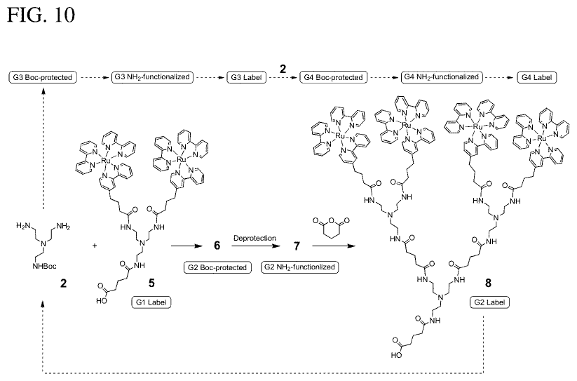

Using the synthetic chemistry practiced in the art, a person of skill is able to prepare the properly functionalized structural building blocks for the ECL labels, and to prepare the ECL labels of the current invention, such as the ECL Label 8 in the Examples and in FIG. 10.

In an embodiment of the present invention, the ECL label comprises at least two transition metal complex ECL luminophores (either same or different). Thus, the detection reagent comprises at least two ECL luminophores and a second chemical/biochemical compound linked covalently. In a further embodiment, the second chemical/biochemical compound is a biological macromolecule. In a further embodiment, the second chemical/biochemical compound is an analyte specific reagent as specified above. In a further embodiment, the second chemical/biochemical compound is an analyte analog/homolog or derivative.

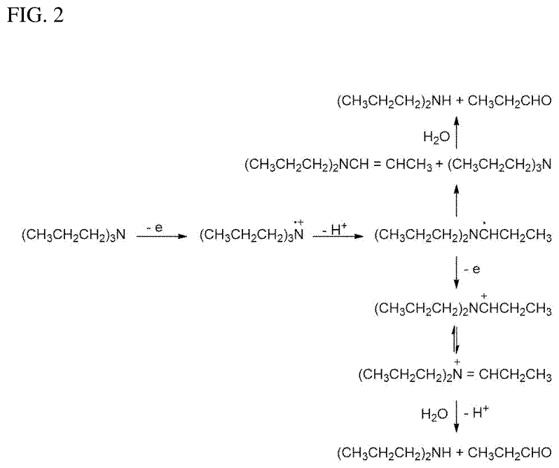

Unlike Ru(bpy)₃²⁺, which undergoes the aforementioned cycle Ru(bpy)₃²⁺ [symbol] Ru(bpy)₃³⁺ [symbol] Ru(bpy)₃^{2+*} [symbol] Ru(bpy)₃²⁺ (Schemes 1-4) and remains undepleted, the co-reactant TPA (or any other analog/homolog of tertiary amine) in the ECL reactions is consumable and is typically added in a large excess over the apparent need for the ECL process in an immunoassay system. Examination of the final products of the electrochemical oxidation led to the findings of the secondary amine, suggesting a carbon-nitrogen bond (C—N bond) cleavage or a dealkylation reaction in the end of the cascade of reactions. Such a dealkylation reaction has been found in almost all aliphatic tertiary amines (e.g., TPA and n-triethylamine) and aromatic amines containing at least one aliphatic C—N linkage. In the case of TPA, the electrochemically derived neutral radical (CH₃CH₂CH₂)₂NC*HCH₂CH₃, while reducing Ru(bpy)₃³⁺ to generate the luminescent excited state Ru(bpy)₃^{2+*} in the ECL system, undergoes either further anodic oxidation or disproportionation to eventually result in the formation of the secondary amine (CH₃CH₂CH₂)₂NH (P. J. Smith and C. K. Mann, J. Org. Chem. 1969, 34, 1821-1825; L. C. Portis et al. J. Org. Chem. 1970, 35, 2175-2178.) as shown in FIG. 2.

Based on the fact that the electrochemical oxidation of tertiary amines leads to dealkylation in three different ways and eventually the product of secondary amines, a properly designed ECL label molecule would incorporates at least one tertiary amine moiety, wherein the amine nitrogen is substituted with two —CH₂—R units, R being an ECL group comprising an ECL luminophore, for example the Ru(bpy)₃²⁺ complex or iridium complex luminophore. Upon dealkylation (i.e., cleavage of a C—N bond) at the electrode at potentials high enough to oxidize Ru(bpy)₃²⁺, the fragments containing the Ru(bpy)₃²⁺ or iridium luminophores, are released. Since dealkylation occurs randomly at one of the three C—N bonds, two ECL groups (each comprising an ECL luminophore) are typically attached to a linking amine N-center for implementing the present invention. The cleavage of any C—N bond would cause detachment of a fragment containing at least one ECL luminophore. FIG. 3 shows four examples of the design, two ruthenium(II) or iridium(III) complex luminophores and a reactive, bioconjugatable (carboxylic or N-succinimidyl carboxylate) group are linked together to a nitrogen atom (an amine N-center).

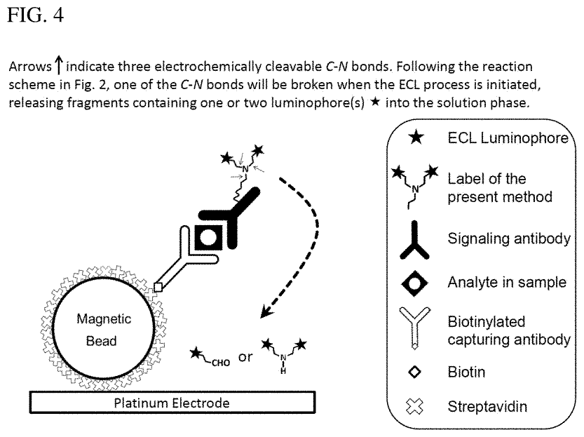

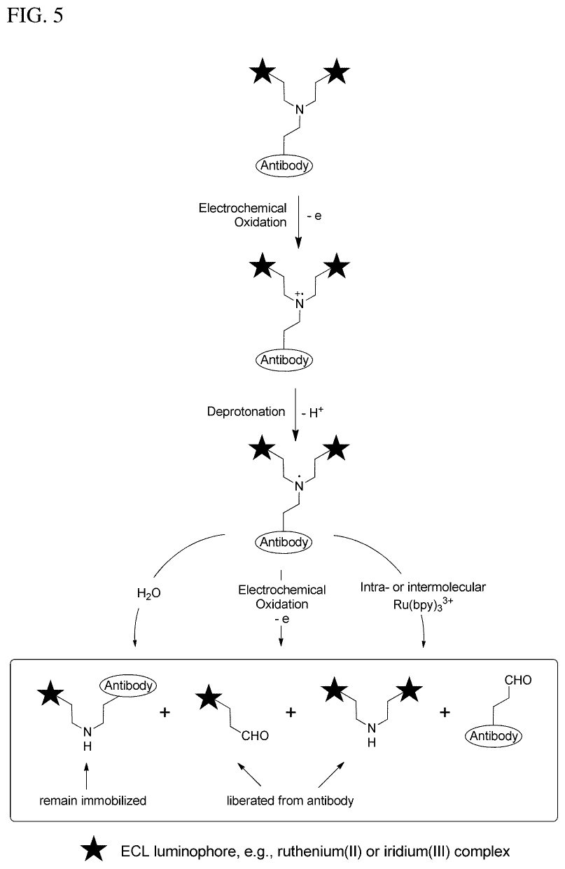

In FIG. 4, a signaling antibody is labeled with an ECL label of the present invention, for example, either one of the labels in FIG. 3, to become a detection reagent. After mixing the sample with the detection reagent, the capturing antibody and the magnetic beads, an antibody/antigen(analyte)/antibody sandwich complex on the surface of a microbead is formed. The arrows indicate three electrochemically cleavable C—N bonds of the ECL label of the present invention. Following the reaction scheme in FIG. 2, one of the three C—N bonds is broken when the ECL process is initiated. As shown in FIG. 5, the electrooxidation at the N-center produces a number of intermediates and eventually releases fragments containing either one or two ECL luminophores into the solution phase.

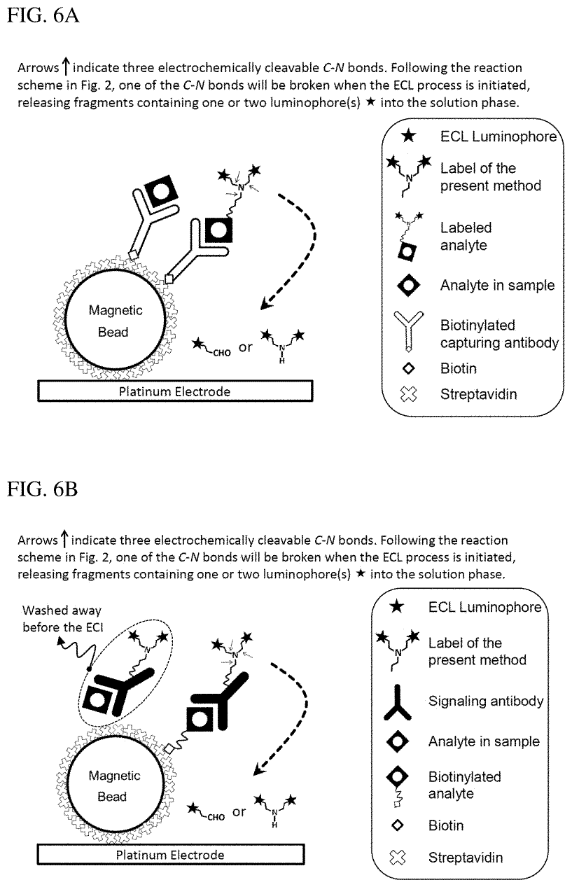

Likewise, in a competitive immunoassay of FIG. 6A, an analyte rather than a signaling antibody is labeled with a label of the present invention to become a detection reagent. The labeled analyte forms a complex with the biotinylated capturing antibody that is immobilized on the surface of a magnetic microbead via surface streptavidin coating. Similarly, one of the three C—N bonds is broken when the ECL process is initiated, releasing fragments containing either one or two ECL luminophores into the solution phase. In a competitive immunoassay the concentration of the analyte in a sample is inversely related to the ECL signal intensity.

FIG. 6B is another form of a competitive immunoassay. The biotinylated analyte competes with the analyte in a sample for binding to the labeled signaling antibody to form the antigen(analyte)/antibody complexes. After magnetic beads are added to the mixture, only the biotinylated analyte/antibody complex is immobilized on the surface of the magnetic beads via strong biotin/streptavidin binding reaction and then remains on the surface of the working electrode in the magnetic field. According to the reaction scheme in FIG. 2, the electrooxidation at the N-center of the label causes the cleavage of one of the three C—N bonds and eventually liberates fragments containing either one or two ECL luminophores.

In one embodiment, the present invention relates to a method of detecting an analyte in a sample comprising the steps of:

a) incubating the sample with a detection reagent to provide an analyte-bound detection reagent,
wherein the sample contains the analyte,
wherein the detection reagent is labeled with one or multiple ECL label(s);
wherein each of the ECL label contains at least one fragment of Formula 1

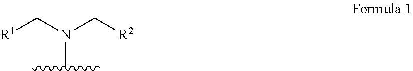

wherein R¹ and R² are the same or different, and each contains an ECL luminophore,
wherein the ECL luminophore is a transition metal complex, and
wherein the analyte-bound detection reagent is immobilized on a solid phase;
b) separating the analyte-bound detection reagent from the detection reagent that is analyte-free and from other unimmobilized species to provide a separated analyte-bound detection reagent, wherein the separated analyte-bound detection reagent is immobilized on the solid phase;
c) contacting the separated analyte-bound detection reagent with an aqueous buffer solution, wherein the aqueous buffer solution comprises at least one tertiary amine;
d) electrochemically triggering oxidation of the ECL luminophores and the tertiary amine, thereby releasing ECL signal; and
e) detecting the electrochemiluminescence signal thereby detecting the analyte.

In one embodiment, the method further comprises an initial step of providing the detection reagent by labeling an analyte specific reagent with one or multiple ECL label(s).

In another embodiment, the method further comprises an initial step of providing the detection reagent by labeling an analyte or analyte analog/homolog/derivative with an ECL label.

In another embodiment, the method further comprises the step of:

f) quantifying the analyte.

In another embodiment, the ECL group R¹ and R² comprises a ruthenium or iridium complex.

In another embodiment, R¹ and R² are the same.

In another embodiment, the tertiary amine is an alkyl tertiary amine. In another embodiment, the tertiary amine is a branched amine. In another embodiment, the tertiary amine is tri-n-propylamine, tri-butylamine, or triethylamine, 2-(dibutylamino)ethanol.

In another embodiment, the analyte specific reagent is a monoclonal antibody.

In another embodiment, the analyte specific reagent is a protein or nucleic acid recognition partner of the analyte or an analog thereof.

In another embodiment, the analyte specific reagent is an analyte analog/homolog/derivative.

In another embodiment, the tertiary amine is in at least a 10-fold excess relative to the concentration of the ECL label. As an example, the tertiary amine is at 10 μM to 1 M, preferably 10 mM to 500 mM.

As discussed above, the present invention provides ECL labels containing electrochemically detachable luminophores that can be liberated from the solid surface in a bioanalytical assay during the signal generation process. The present invention also provides a method of incorporating tertiary amine structural unit(s) into an ECL label molecule to achieve electrochemical detachment of the ECL luminophore(s) during the ECL generation process.

In one aspect, the ECL label used in the present invention contains one fragment of Formula 1. As an example, the two ECL groups and a bioconjugatable moiety are linked through a trialkyl amine N-centers to form the invented label of Formula 2

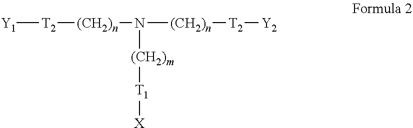

wherein, Y₁ and Y₂, same or different, are each an ECL luminophore (or a substituted luminophore or a group containing an ECL luminophore); X is a carboxyl, N-succinimidyl carboxylate, sulfo-N-succinimidyl carboxylate, phosphoramidite, isothiocyanato, formyl, hydrazino, hydroxyl, or maleimido; T₁ and T₂ are independently each an alkylene, alkenylene, —O—C₁₋₁₀—, —C₁₋₁₀—O—C₁₋₁₀—, —CONH—C₁₋₁₀—, —C₁₋₁₀—CONH—, —NHCO—C₁₋₁₀—, an aromatic ring; m and n are independently each an integer between 1 and 10. In a preferred embodiment, the ECL luminophore or the substituted luminophore is ruthenium and iridium containing complex.

Y₁ and Y₂ are independently each a ruthenium (II) or iridium(III) complexes as described above. Examples of the ruthenium (II) complexes include polydiimine complexes. Examples of the iridium(III) complexes include cyclometalated iridium(III) complexes. Because the current invention concerns ECL labels bearing multiple ECL groups, the structural features of the ruthenium (II) or iridium(III) complexes within each ECL group are not particularly limited.

In another aspect, the invention provides an ECL label containing multiple fragments of Formula 1, and thus multiple ECL luminophores. Specifically, two types of such labels have features: (1) a branched skeleton bearing only peripherial N-centers, each of which is linked to two ECL luminophores (Type-I), and (2) a branched skeleton composed of hierarchically levelled N-centers as branch joints and only the peripherial N-centers are each linked to two ECL luminophores (Type-II). In a preferred embodiment, the ECL luminophores are ruthenium(II) and iridium(III) containing complexes described above.

The Type-I ECL labels of the present invention can be prepared by linking the labels of Formula 2 (exemplified in FIG. 3) to a branched skeleton, such as a modified pentaerythritol skeleton (described in Anal. Chem., 2003, 75, 6708-6717), an α-polylysine or a peptides with multiple lysine residues in the sequence. Upon electrochemical dealkylation during the ECL process, the detection reagent labeled with the ECL labels of Formula 2 (also exemplified in FIG. 3) or Type-I will be decomposed to free fragments containing either one or two ECL luminophore(s) shown in the bottom box of FIG. 5.

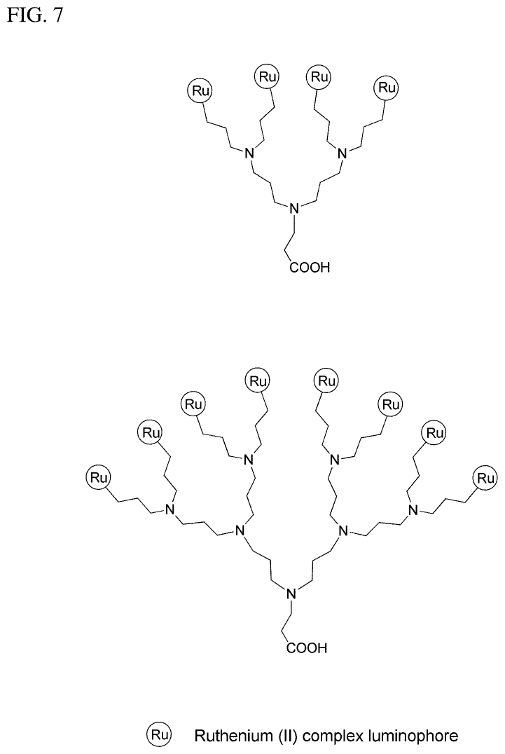

The Type-II ECL labels are typically molecules with a dendritic structure. FIG. 7 illustrates two embodiments of the Type-II ECL labels of the current invention, wherein a dendritic skeleton composed of hierarchically levelled N-center as branch joints and only the peripheral N-centers are each linked to two ruthenium complex units as the ECL luminophores.

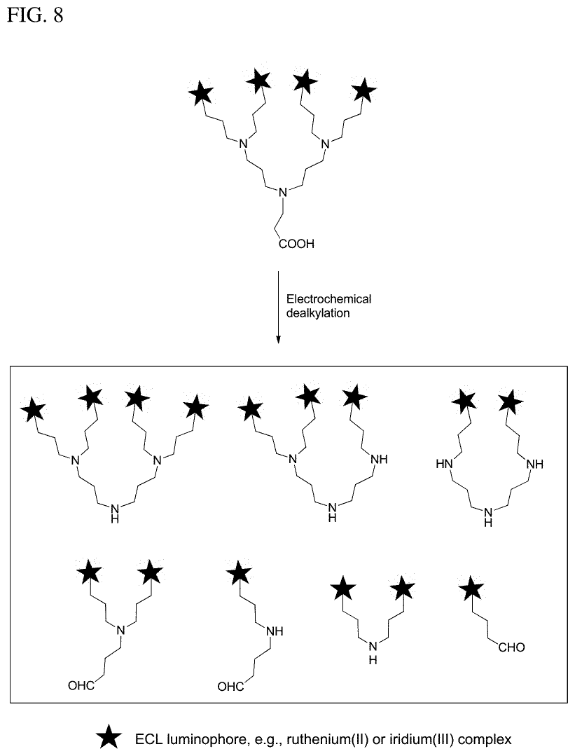

Upon electrochemical dealkylation during the ECL process, the detection reagent labeled with the Type-II ECL labels will give rise to more complicated fragmentation in ECL immunoassay. Since each of the hierarchically levelled N-center would undergo dealkylation, a number of different fragments, which contain different numbers of luminophores (as depicted in FIG. 8), would be liberated from the solid surface and migrate into the solution phase for ECL generation. Furthermore, the larger fragments containing trialkyl N-center(s) in solution phase continue to follow the dealkylation process and give rise to smaller fragments.

The Type-II ECL label molecules in FIG. 7 can be synthesized through either a divergent or a convergent approach, commonly employed in the syntheses of dendrimers (see F. Vögtle, Topics in Current Chemistry Vol. 197: Dendrimers, Springer, 1998).

One embodiment is based on a divergent-iterative pathway leading to dendrimers with a plurality of amino (—NH₂) group at the periphery of the dendrimers. An conventional ECL label, such as one of those ruthenium(II) or iridium(III) complexes with either carboxyl or N-succinimidyl carboxylate moiety (FIG. 1) can then react with the amino (—NH₂) groups to form the ECL labels of the present invention. For higher generation dendrimers, a large number of reactive sites (e.g., —NH₂) at the periphery may cause incomplete reaction and thus results in structural imperfection. Nevertheless, the minor structural defects in a higher generation dendrimer do not compromise its use as an ECL label in this invention.

The convergent-iterative pathway starts the synthesis from the periphery inwards to a focal point. The ECL luminophores were first incorporated into a branch structural unit. In one embodiment based on a convergent synthetic strategy, N¹, N¹-bis(2-aminoethyl)ethane-1,2-diamine is employed as the trialkyl amine N-center to be incorporated into label molecules of this invention. An ECL conventional label, such as one of those ruthenium(II) or iridium(III) complexes with either carboxyl or N-succinimidyl carboxylate moiety (FIG. 1) is used for incorporating the ECL luminnophores into the labels of the present invention.

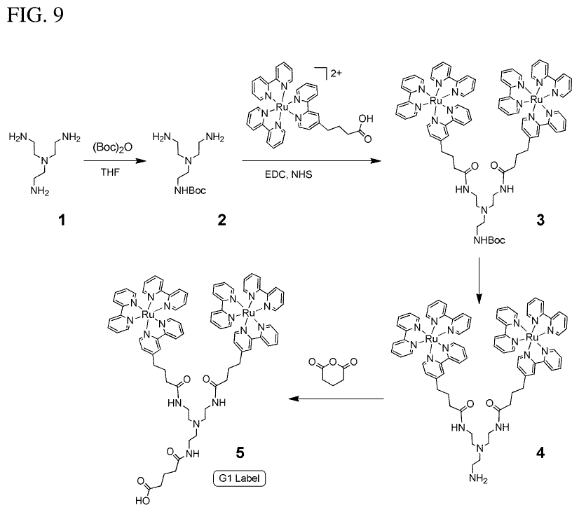

FIGS. 9 and 10 shows how the hierarchically different amine N-centers and a plurality of metal complex (e.g., Ru(bpy)₃²⁺) luminophores are linked together in the convergent synthesis to form Type-II dendritic labels with the invented features. G1 label of Formula 2 is first prepared to contain two luminophores in this invention (FIG. 9). By an iterative method, higher generations of dendritic labels (G2, G3, G4, G5 and so on) with more ECL luminophores may be prepared as shown in FIG. 10.

In another embodiment, ECL labels of this invention can be constructed with both the features of aforementioned Type-I and Type-II labels. For example, replacing the G1 labels of Formula 2 (exemplified in FIG. 3) with G2, G3 or G4 labels, molecules sharing both Type-I and Type-II features can be synthesized by an esterization of the carboxylic labels (such as G2, G3 and G4 shown in FIG. 10) with a modified pentaerythritol skeleton described in Anal. Chem. 2003, 75, 6708-6717. Similarly, in another embodiment, peptides with multiple lysine residues in the sequence or α-polylysine can be used as the branched skeleton for incorporating multiple amine interlinked ECL luminophores (in G2, G3 and G4 labels) by forming an amide bond between the labels in FIG. 10 and the branched skeleton.

By detaching the immobilized luminophores during the ECL process, the present invention solves the problem associated with limited accessibility of the densely assembled luminophores in the prior art (M. Zhou et al., Macromolecules 2001, 34, 244-252; M. Zhou et al., Anal. Chem. 2003, 75, 6708-6717; M. Staffilani, et al., Inorg. Chem. 2003, 42, 7789-7798) and thus rejuvenates the single-site multi-labeling method previously disclosed in US 2005/0059834 A1 and CA 2481982 A1. As aforementioned, the liberated luminophores, or free fragments containing luminophores, are more accessible and more reactive than the immobilized or bound luminophores. Therefore, higher levels of ECL signal could be generated from the homogeneous solution. Moreover, to our surprise, the ECL decays more slowly with the time during the ECL emission process.

The examples below explain the spirit of the invention. That is—incorporating one or more amine structural unit(s) into a label molecule so that one or more C—N bond(s) of N-center(s) can be broken upon electrochemical dealkylation and, thus releasing one or more immobilized ECL luminophore into solution phase for enhanced ECL signal.

### EXAMPLES

### Example 1—Synthesis of an Amine Skeleton Bearing Two Diimine Ligands

### 1. Synthesis of N,N′-(((2-aminoethyl)azanediyl)bis(ethane-2,1-diyl))bis(4-([2,2′-bipyridin]-4-yl)butanamide)(10)

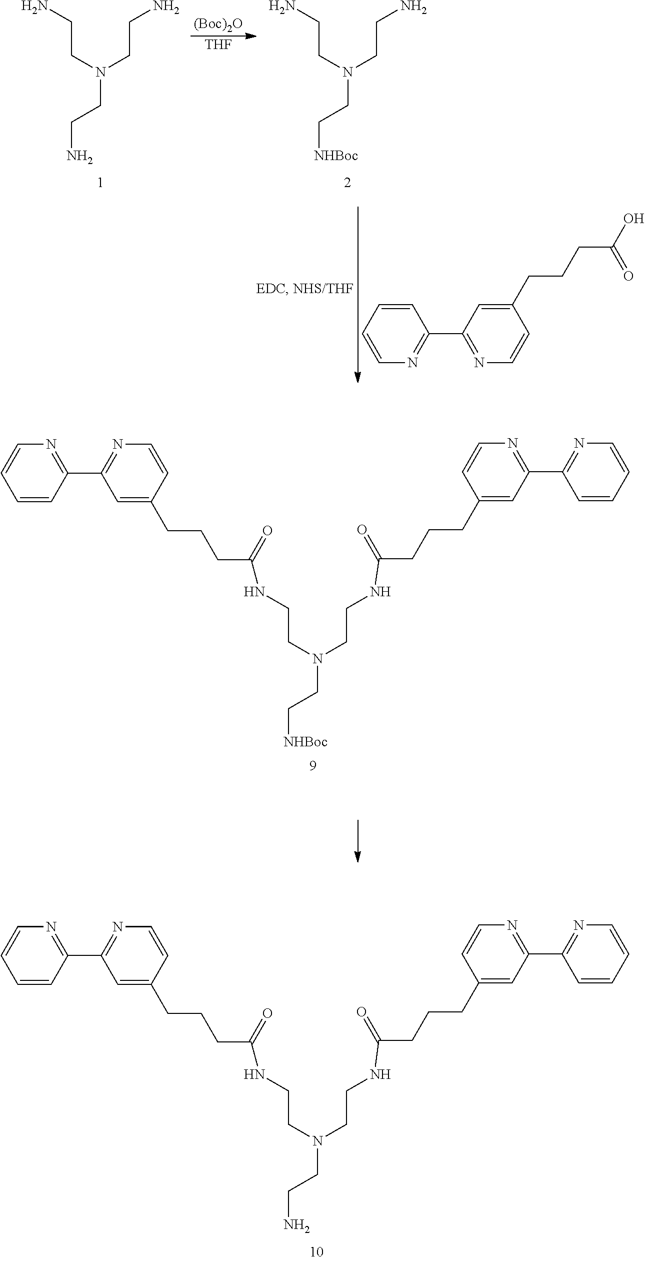

10.24 g (70 mmol) of compound 1 was dissolved in 150 mL of anhydrous THF at room temperature. To this solution was slowed dropped (Boc)₂O/THF (16.4 g, 75 mmol (Boc)₂O in 150 mL Dioxane). The mixture was kept stirring overnight and roto-evaporated. The coarse product was extracted with ethyl acetate, washed with saturated NaCl aqueous solution and dried with anhydrous Na₂SO₄. The column chromatography over silica gel yielded 16.5 g of 2 (96%). ¹H NMR (CDCl₃, 400M Hz) δ 5.31 (1H), 3.16 (2H), 2.73 (4H), 2.50 (6H), 1.41 (9H), 1.31 (4H).

2.42 g (10 mmol) of 4-([2,2′-bipyridin]-4-yl)butanoic acid was dissolved in 40 mL of anhydrous DMF. To this solution was added N-hydroxysuccinimide (NHS, 2.3 g, 20 mmol) and N,N′-dicyclohexylcarbodiimide DCC (4.2 g, 20.3 mmol). After 4 hours, the cloudy mixture was filtered. To the filtrate was added 1.1 g (4.5 mmol) of 2. The mixture was kept stirring overnight at room temperature. Roto-evaporation of the liquid yielded yellow coarse product. The column chromatography over silica gel (20:1 methanol/dichloromethane elution) yielded 2.7 g of white solid 9 (87%).

Dissolve 3 (2.7 g) in methanol/water(1:2) solution and cool the solution in an ice/water bath for 30 min. To this cooled solution was dropped 4 mL of concentrated hydrocholoric acid. The mixture was kept stirring in the ice/water bath for one hour and, at room temperature, for another three hours. Roto-evaporation of the liquid yielded white solid 10 (chloride).

### 2. Synthesis of tert-butyl 5-((2-(bis(2-(4-([2,2′-bipyridin]-4-yl)butanamido)ethyl)amino)ethyl)amino)-5-oxopentanoic acid (11)

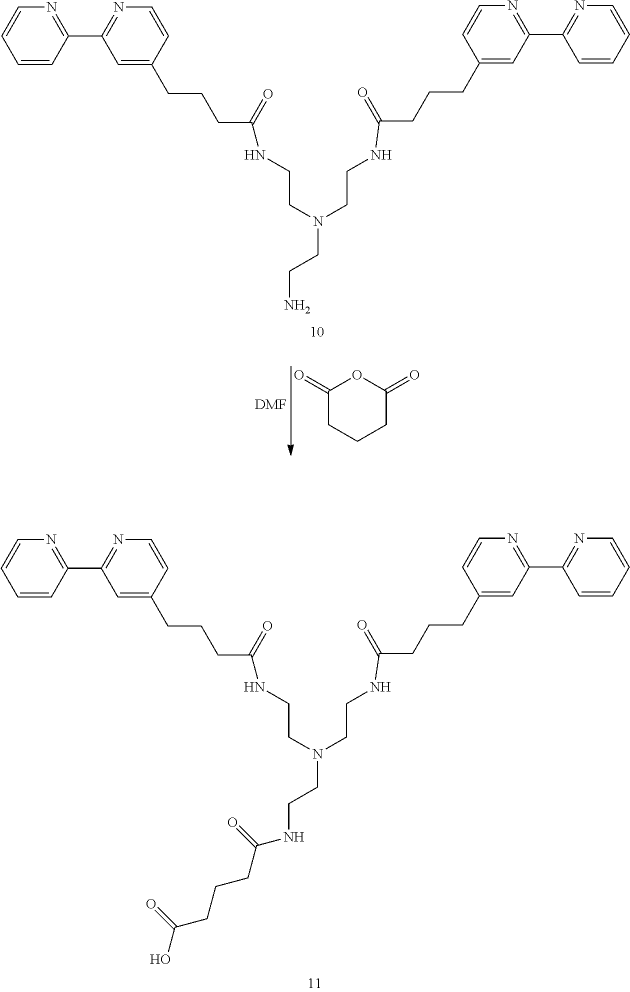

2.3 g (3.87 mmol) of 10 and 515 mg (4.51 mmol) of glutaric anhydride were mixed in 15 mL of DMF. The reaction mixture was heated to 50° C. for 6 hours under nitrogen. After the reaction was complete, the solvent DMF was roto-evaporated. The residue was column (silica gel) purified and subsequently dried in vacuo to afford 2.1 g white powder 11 (76%).

### Example 2—Synthesis of a Bis(diimine)ruthenium(II) Complex

### 3. Synthesis of (p-cymene)(bathophenanthroline disulfonate sodium)RuCl₂ (12)

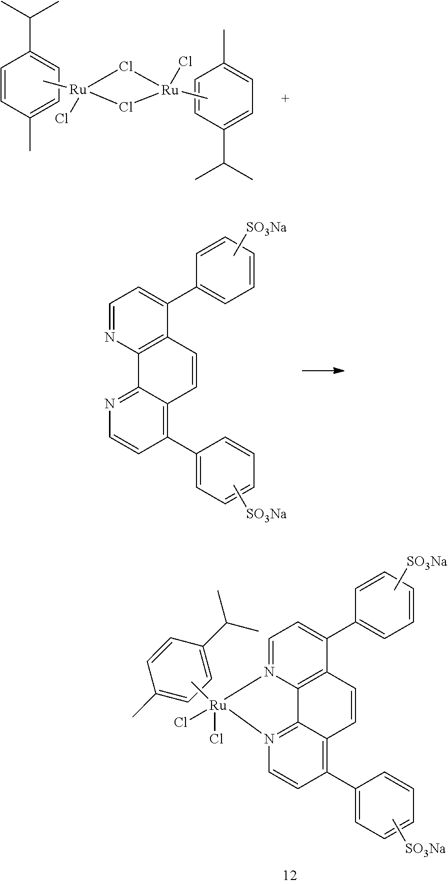

[(p-cymene)RuCl₂]₂ (0.806 g, 1.316 mmol) in 150 mL of methanol and bathophenanthroline disulfonate sodium (1.412 g (2.632 mmol) in 75 mL of water were mixed and refluxed under argon for four hours. The solvents were roto-evaporated and the yellow solid (2.2 g) was used without further purification for further use.

### 4. Synthesis of (2,2′-bipyridine)(bathophenanthroline disulfonate sodium) ruthenium(II) dichloride complex (13)

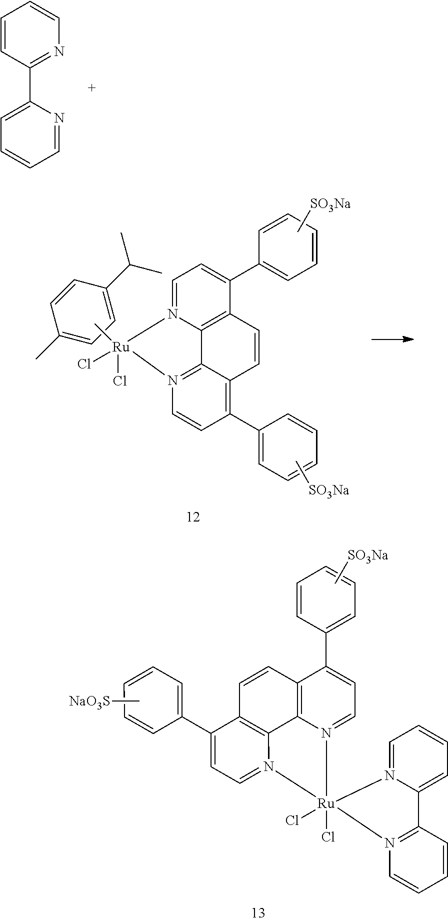

2,2′-bipyridine (0.355 g, 2.27 mmol) and 12 (1.785 g, 2.12 mmol) were mixed with 0.772 g (18.2 mmol) of lithium chloride in 30 mL of DMF. The solution was refluxed under nitrogen for 3.5 hours. After cooling to room temperature, the dark purple solution was roto-evaporated and the solid was re-dissolved in methanol. The column chromatography over silica gel yield 1.75 g dark purple solid (13).

### Example 3—Synthesis of an ECL Label

### 5. Synthesis of a Label with Two Luminophores Linked to an Amine Unit (14)

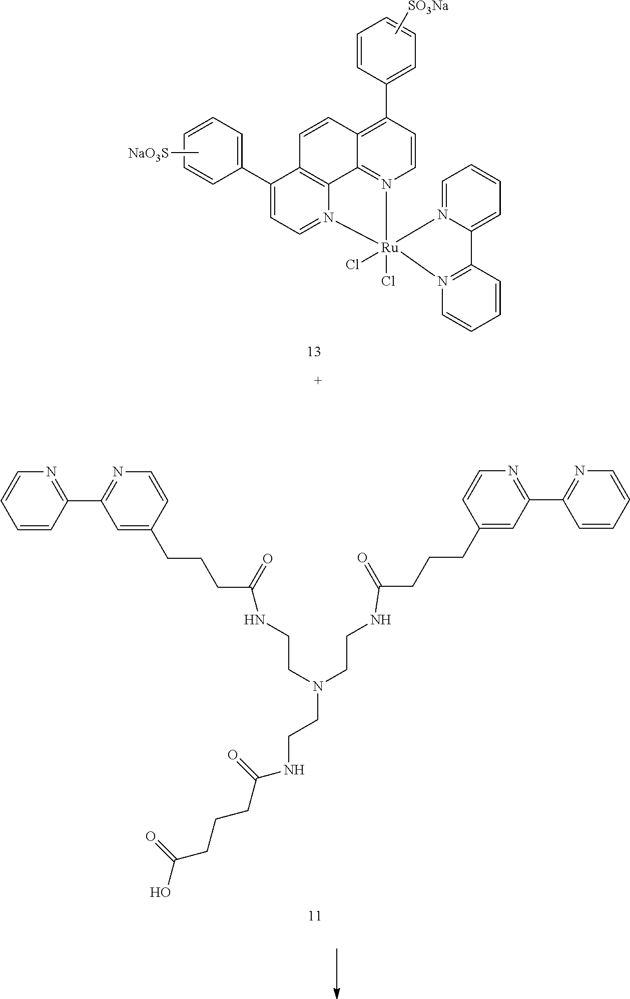

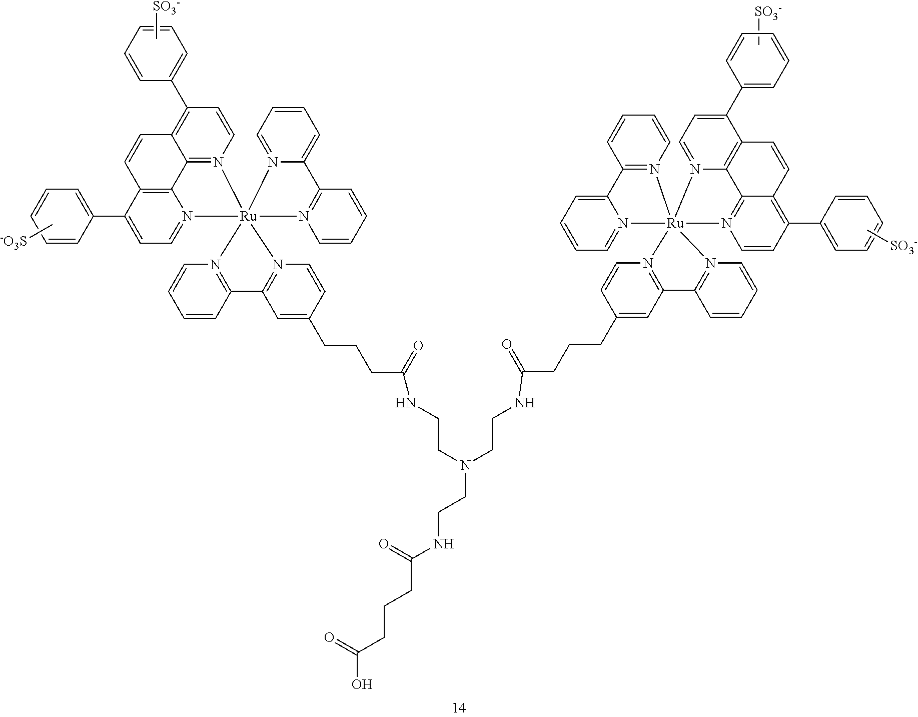

600 mg (0.69 mmol) of 13 and 213 mg (0.3 mmol) of 11 were dissolved in 40 mL of methanol/water (3:1) and the solution was refluxes for 3 hours under nitrogen. The color changed from dark purple into bright orange during this process. The reaction solution was filtered and concentrated to 10 mL followed by column chromatography over silica gel using methanol/water (2:1). The target compound was obtained from roto-evaporation and dried in vacuo. Yield 520 mg (76%).

### Example 4—Antibody Labeling with an Aforementioned Label

### 6. Labeling an α1-Fetoprotein (AFP) Antibody (Signaling Antibody) with Label 14 to Form Detection Reagent 15

3.4 mg (1.5 μmol) of 14 was dissolved in 500 L of MES buffer (0.1 mol L⁻¹, pH=4.7) at a concentration of 3.0 mmol L⁻¹. 1.5 mg (7.82 mol) of 1-ethyl-3-[3-dimethylaminopropyl]carbodiimide hydrochloride (EDC) and 4.5 mg (20.7 μmol) of N-Hydroxysulfosuccinimide sodium salt (sulfo-NHS) were added to this solution to obtain a concentration of approximately 15.6 mmol L⁻¹ EDC and 41.4 mmol L⁻¹ sulfo-NHS. The solution was shaken for 10 minutes at room temperature. 1.0 L (14 μmol) of 2-mercaptoethanol was added to the reaction solution (final concentration of 30 mmol L⁻¹). After 5 minutes at room temperature, 10 L (containing 30 nmol of 14) of this incubated solution was added to 500 L of AFP antibody (0.9 mg/mL, approximate 3 nmol of antibody, the challenge ratio 10:1) PBS (0.1 mol L⁻¹, pH=7.4). It was mixed and incubated for 2 hours at room temperature.

The solution (about 500 μL) obtained above was loaded onto the PD-10 column (packed with Sephadex G-25 medium, from GE Healthcare Life Sciences) that was pre-equilibrated with PBS. Two yellow bands formed during the separation. The first eluted band, corresponding to the labeled antibody, i.e., detection reagent 15, was collected (about 750 μL).

To determine the actual label to protein ratio (the conjugation ratio), Bradford or BCA protein assay was used to quantify the AFP antibody. The absorbance of label 14 at 455 nm was correlated to the label quantity based on the extinction coefficient of the label 14. Under the conditions described above, a detection reagent 15 molecule contained about 5.5 luminophores of label 14.

### 7. Labeling an AFP Antibody with Ru(2,2′-bipyridine)(bathophenanthroline disulfonate) [4-(2,2′-bipyridin-4-yl)butanoic Acid] (the Electronically Neutral Label Shown in FIG. 1, Denoted as Ref) to Form Detection Reagent 16

2.5 mg (2.5 μmol) of Ref was dissolved in 500 L of MES buffer (0.1 mol L⁻¹, pH=4.7) at a concentration of 5.0 mmol L⁻¹. 1.0 mg (5.2 mol) of EDC and 3.0 mg (13.8 μmol) of sulfo-NHS were added to this solution to obtain a concentration of approximately 10 mmol L⁻¹ EDC and 27 mmol L⁻¹ sulfo-NHS. The solution was shaken for 10 minutes at room temperature. 0.7 L (10 mol) of 2-mercaptoethanol was added to the reaction solution (final concentration of 20 mmol L⁻¹). After 5 minutes at room temperature, 8.0 L (containing 40 nmol of Ref) of this incubated solution was added to 500 L of AFP antibody (1.2 mg/mL, approximate 4 nmol of pure AFP antibody, the challenge ratio 10) PBS (0.1 mol L⁻¹, pH=7.4). It was mixed and incubated for 2 hours at room temperature.

The solution (about 0.5 ml) obtained above was loaded onto the PD-10 column that was pre-equilibrated with PBS. Two yellow bands formed during the separation. The first eluted band, corresponding to the labeled antibody, i.e., detection reagent 16, was collected (about 0.75 ml). The conjugation ratio of label Ref to the antibody was determined to be 6.1:1.

### 8. Biotinylation of Capturing AFP Antibody to Form Biotinylated Antibody 17

To 2 mL of CBS buffer (pH 9.5) containing 2.1 mg of desalted AFP capturing antibody was added an aliquot (15 L) of 20 mM of NHS-LC-biotin (MW 454.54) DMF solution and the mixture was incubated for one hour followed by the dialysis of total 20 hrs in PBS buffers. The final concentration of the biotinylated capturing AFP antibody 17 was determined to be 1.72 mg/mL by a BCA assay.

### Example 4—Immunoassay

### 10. Sandwich Immunoassay Using AFP Detection Reagent 15 (Antibody Labeled with 14 of the Present Invention) in Comparison with the AFP Detection Reagent 16 (Antibody Labeled with Ref)

The ruthenylated AFP signaling antibodies 15 or 16, and the biotinylated capturing AFP antibody 17 were diluted with PBS buffer (pH 6.0) to 12 μg mL⁻¹ and 4.5 μg mL⁻¹, respectively. Dynabeads® M-280 coated with streptavidin was the magnetic media for capturing the biotinylated antibody/antigen/ruthenylated antibody immunocomplex.

The solutions of 1000 μl of AFP (analyte) at different concentrations in calf serum were mixed with 20 μl of 4.5 μg mL⁻¹ of biotinylated capturing AFP antibody 17 in PBS, 20 μl of 12 g mL⁻¹ of labeled AFP antibody 15 or 16. Each mixture was incubated for 20 minutes at room temperature to form the sandwich immunocomplex 18 or 19, respectively. A suspension of 20 μl streptavidin-coated Dynabeads® M-280 (200 μg beads, pre-washed three time by PBS) was added to the above solution of sandwich immunocomplex 18 or 19. This suspension of total volume of 1060 μl was incubated for 20 minutes with constant gentle rotation at room temperature. The immunocomplex loaded Dynabeads 20 or 21 were separated with an external magnet for 2 minutes while other unreacted species were removed by washing the beads 20 or 21 with 1000 μl PBS containing 0.1% BSA for four times. After the final washing step, the beads 20 or 21 were resuspended in 1000 μl of PBS to the concentration of 200 μg mL⁻¹ of beads 20 or 21 loaded with antibody/antigen/antibody sandwich immunocomplex 18 or 19, respectively. 100 μl of the above suspension was injected into the three-electrode measuring cell with a photomultiplier tube above the working electrode and a movable magnet underneath the working electrode. A potential step (1.4 V vs. Ag/AgCl) was employed to oxidize the TPA and the ruthenium(II) luminophore, which were immobilized on the magnetic beads via the antibody/antigen/antibody sandwich, to generate ECL through the oxidative-reduction process in a phosphate buffer (ProCell from Roche Diagnostics, pH 6.8, 0.18 mol L⁻¹ tripropylamine solutions). After each measurement, the measuring cell was cleaned and the electrode was regenerated electrochemically as described in the U.S. Pat. Nos. 5,538,687 and 6,599,473B1.

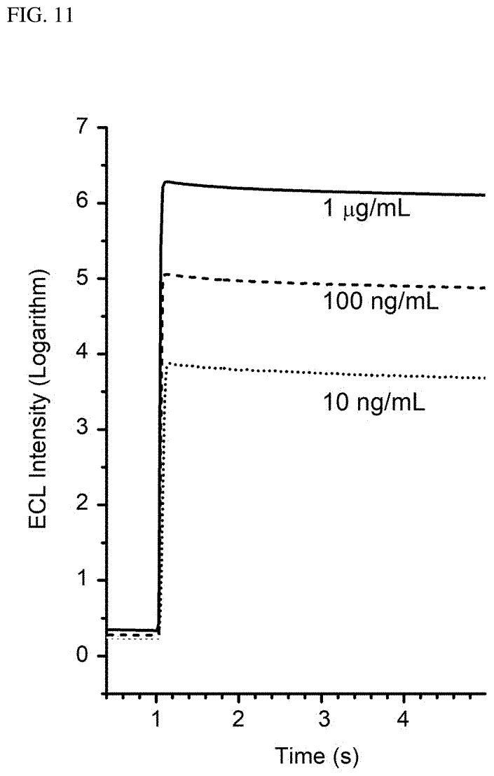

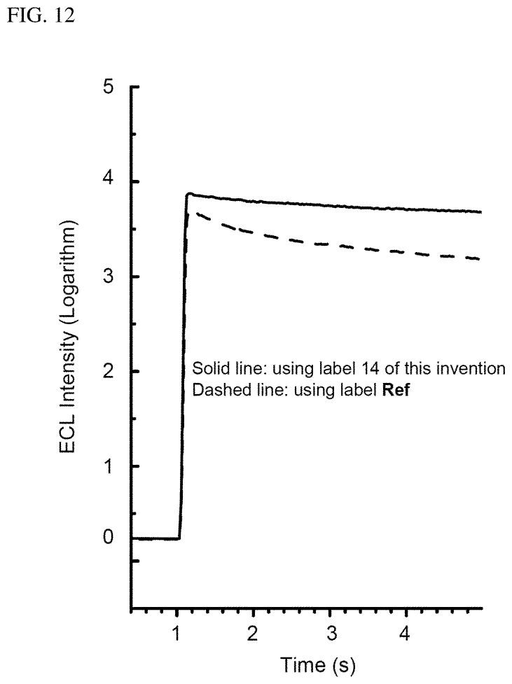

The ECL signal decay in each immunoassay experiment was plotted in FIGS. 11 and 12. Compared with the Ref-labeled antibody 16, the detection reagent 15 (labeled with the label of this invention) demonstrated more intense and more stable ECL on the measurement time scale (four seconds in FIG. 12).

These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

What is claimed is:

1. A method of detecting an analyte in a sample, comprising the steps of
a) incubating the sample with a detection reagent to provide an analyte-bound labeled detection reagent,
wherein the sample contains the analyte,
wherein the detection reagent is labeled with one or multiple ECL label(s), wherein the ECL label contains at least one fragment of Formula 1: 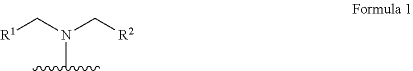
wherein R¹ and R² are the same or different, and each contains an ECL luminophore,
wherein the ECL luminophore is a transition metal complex and
wherein the analyte-bound labeled detection reagent is immobilized on a solid phase;
b) separating the analyte-bound detection reagent from the analyte-free detection reagent and other unimmobilized species to provide a separated analyte-bound labeled detection reagent, wherein the separated analyte-bound labeled detection reagent is immobilized on the solid phase;
c) contacting the separated analyte-bound labeled detection reagent with an aqueous buffer solution, wherein the aqueous buffer solution comprises at least one tertiary amine;
d) electrochemically triggering oxidation of the ECL luminophore and the tertiary amine, thereby releasing ECL signal; and
e) detecting the ECL signal thereby detecting the analyte.

2. The method of claim 1, further comprising an initial step of providing the detection reagent by labeling an analyte specific reagent with one or multiple ECL label(s).

3. The method of claim 1, further comprising the step of
f) quantifying the analyte.

4. The method of claim 1, wherein the ECL label comprises a ruthenium or iridium complex.

5. The method of claim 1, wherein R¹ and R² are the same.

6. The method of claim 1, wherein the tertiary amine is an alkyl tertiary amine.

7. The method of claim 6, wherein the alkyl tertiary amine is a branched amine.

8. The method of claim 1, wherein the tertiary amine is tri-n-propylamine, tri-butylamine, triethylamine, 2-(dibutylamino)ethanol.

9. The method of claim 2, wherein the analyte specific reagent is a monoclonal antibody, a protein or nucleic acid recognition partner of the analyte or an analog thereof, or an analog/homolog/derivative of the analyte.

10. The method of claim 1, wherein the tertiary amine is in at least a 10-fold excess relative to the concentration of the ECL label.
